(19) United States
(12) Patent Application Publication
(10) Pub. No.: US 2003/0194433 A1
(43) Pub. Date: Oct. 16, 2003

(54) Antimicrobial compositions, methods and articles employing singlet oxygen- generating agent

(75) Inventors: Robert D.P. Hei, Baldwin, WI (US); Stephan M. Hubig, Maplewood, MN (US); Matthew J. Finley, Shakopee, MN (US)

Correspondence Address: David R. Cleveland, David R. Cleveland, P.A., Suite E-1324 First Nat'l Bank Bldg., 332 Minnesota Street, Saint Paul, MN, 55101, US

(73) Assignee: Ecolab

(21) Appl. No.: 10/097,232

(22) Filed: Mar. 12, 2002

(51) Int. Cl. A61K 31/555; A61K 31/542; A61K 31/473; A61K 31/365

(52) U.S. Cl. 424/465; 514/297; 514/185; 514/410; 514/454; 514/224.8

(57) ABSTRACT

Antimicrobial compositions containing a singlet oxygen-generating agent and a lubricant exhibit very effective antimicrobial activity against a variety of organisms, including Gram-positive bacteria, Gram-negative bacteria, fungi and yeast. The compositions have particular utility as beverage container lubricants. The passage of a container along a conveyor is antimicrobially lubricated by applying a lubricant to at least a portion of the container-contacting surface of the conveyor or to at least a portion of the conveyor-contacting surface of the container and generating singlet oxygen in situ. This method provides effective control of microbes on a beverage conveyor line, at rates comparable to purely chemical biocidal systems.

### TECHNICAL FIELD

This invention relates to lubricants. The invention also relates to methods for conveying articles, conveyor systems and containers wholly or partially coated with such lubricants.

### BACKGROUND

In commercial container filling or packaging operations, the containers typically are moved by a conveying system at very high rates of speed. Typically, copious amounts of aqueous dilute lubricant solutions (based, for example, on soap solutions or on fatty acid amines) are applied to the conveyor or containers using spray or pumping equipment. Conveyor lubricants permit high-speed operation of the conveyor and limit marring of the containers or labels, but can have significant disadvantages. For example, some conveyor lubricants can promote the growth of microbes, leading to possibly unhealthy conditions on the conveyor line or in the filled containers.

Although not involving conveyor lubricants, U.S. Pat. No. 4,243,539 describes an oil antioxidant made by reacting singlet oxygen and a hindered bis-p-methylphenol. In Example 1 of the '539 patent, the reaction product is formed using oil as the reaction medium.

Moore, P., “Lethal Weapon”, New Scientist, 158, No. 2130, pp. 40-43 (Apr. 18, 1998) describes the use of photosensitizers such as porphyrins and phthalocyanines to generate singlet oxygen in a cancer treatment technique known as photodynamic therapy (PDT). The New Scientist article also discusses the possibility of using photosensitizers and singlet oxygen for other applications.

### SUMMARY OF INVENTION

The present invention provides, in one aspect, antimicrobial compositions comprising a singlet oxygen-generating agent and a lubricant. The compositions of the invention have particular utility as beverage container lubricants, and have very effective antimicrobial activity against a variety of organisms, including Gram-positive bacteria, Gram-negative bacteria, fungi and yeast.

In another aspect, the invention provides a method for antimicrobially lubricating the passage of a container along a conveyor comprising applying a lubricant to at least a portion of the container-contacting surface of the conveyor or to at least a portion of the conveyor-contacting surface of the container and generating singlet oxygen in situ. The method provides effective control of microbes, at rates comparable to purely chemical biocidal systems.

In a further aspect, the invention provides a lubricated conveyor or container, having a lubricant coating on a container-contacting surface of the conveyor or on a conveyor-contacting surface of the container, wherein the coating comprises a singlet oxygen-generating antimicrobial agent.

### BRIEF DESCRIPTION OF THE DRAWING

FIG. 1 illustrates in partial cross-section a side view of a plastic beverage container and conveyor partially coated with an antimicrobial composition of the invention.

### DETAILED DESCRIPTION

The compositions of the invention are “antimicrobial”. As used in this invention, the term “antimicrobial composition” refers to a composition having the ability to destroy or suppress the growth of microorganisms. In most instances, using the procedure set out in Germicidal and Detergent Sanitizing Action of Disinfectants, Official Methods of Analysis of the Association of Official Analytical Chemists, paragraph 960.09 and applicable sections, 15th Edition, 1990 (EPA Guideline 91-2), the antimicrobial compositions of the invention provide greater than a 90% reduction (1-log order reduction), preferably greater than a 99% reduction (2-log order reduction), more preferably greater than a 99.99% reduction (4-log order reduction), and most preferably greater than a 99.999% reduction (5-log order reduction) in one or more targeted species of interest (e.g., the bacteria S. aureus, P. aeruginosa, E. coli or S. typhi, or the fungus Aspergillus sp.) within 30 minutes at ambient temperature. Preferably, the antimicrobial compositions of the invention also provide greater than a 99% reduction (2-log order reduction, more preferably greater than a 99.99% reduction (4-log order reduction), and most preferably greater than a 99.999% reduction (5-log order reduction) in the population of two or more targeted species of interest under such conditions. Because in their broadest sense these definitions for antimicrobial activity are different from some current governmental regulations, the use in connection with this invention of the term “antimicrobial composition” is not intended to indicate compliance with any particular governmental standard for antimicrobial activity.

The compositions and methods of the invention employ singlet oxygen to reduce or control microbial levels in an antimicrobial composition. Typically, such reduction or control of microbial levels will take place while the antimicrobial composition is in lubricious contact with at least two sliding surfaces. As used in this invention, the term “in situ” refers to the generation of singlet oxygen (or addition thereof to the antimicrobial composition) while the antimicrobial composition is in contact with such sliding surfaces. Depending in part on the generation scheme employed, singlet oxygen typically has a relatively short half-life. If suitable care is taken to exclude moisture and other species that could cause early decay of the singlet oxygen and if appropriate storage and delivery measures are taken, singlet oxygen might be generated separately from the antimicrobial composition, then collected, stored or transported as needed and added to the lubricant portion of the antimicrobial composition as required. However, in general it will be much easier and thus preferred to generate singlet oxygen within the antimicrobial composition at or near the point of use on the targeted species.

The antimicrobial composition can be a solid or liquid. If a solid, the antimicrobial composition can have a powdered form, or if desired can be shaped or molded into a suitable pellet, block or other convenient shape, e.g., for use in a dispenser. If in liquid form, the antimicrobial composition can be aqueous (that is, primarily containing water) or non-aqueous (that is, primarily containing a liquid or liquids other than water).

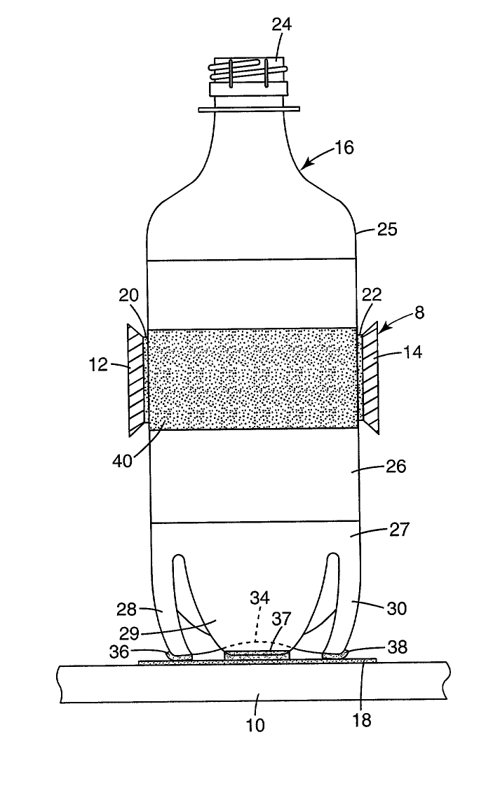

The invention is further illustrated in FIG. 1, which shows in partial cross-sectional view a conveyor 8 having belt 10 and conveyor chute guides 12, 14. Beverage container 16 rides on belt 10 between chute guides 12, 14. The container-contacting portions of belt 10 and chute guides 12, 14 are coated with thin layers 18, 20 and 22 of an antimicrobial composition of the invention. Container 16 is constructed of blow-molded PET, and has a threaded end 24, side 25, label 26 and base portion 27. Base portion 27 has feet 28, 29 and 30, and crown portion (shown partially in phantom) 34. Thin layers 36, 37 and 38 of the antimicrobial composition cover the conveyor-contacting portions of container 16 on feet 28, 29 and 30, but not crown portion 34. Thin layer 40 of the antimicrobial composition also covers the conveyor-contacting portions of container 16 on label 26. The antimicrobial composition kills microbes that otherwise might accumulate on conveyor 8 or containers 16.

The compositions of the invention contain at least one singlet oxygen-generating agent, and the methods of the invention employ singlet oxygen. In the presence of singlet oxygen, microbial levels are rapidly reduced. Singlet oxygen can be generated in a variety of ways, including via photochemical, chemical or enzymatic reactions, or via electric discharge. Photochemical generation is preferred. A particularly convenient photochemical generation mechanism involves the exposure of a suitable sensitizer (sometimes also referred to as a photo-activating dye) to light in the presence of molecular oxygen according to the following reactions:

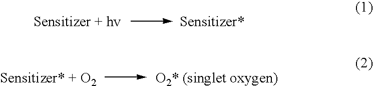

In this mechanism, energy transfer from an excited state (typically a triplet state) of the sensitizer to molecular oxygen generates singlet oxygen molecules. This method is advantageous in that singlet oxygen can be generated within the antimicrobial composition rather than being generated separately. However, the excited state of the sensitizer typically has a relatively short lifetime, and that will have a bearing on the efficacy of this method. The sensitizer preferably has a sufficiently long lifetime in its excited state so that sufficient molecular oxygen will be encountered and sufficient singlet oxygen generated by the excited sensitizer. Preferably this lifetime is at least about 2 microseconds, more preferably at least about 10 microseconds, and most preferably at least about 100 microseconds.

A wide variety of sensitizers can be employed in photochemical generation of singlet oxygen, including aromatic carbonyl compounds (e.g., acetonaphthone, acetophenone and benzophenone), condensed aromatic compounds (e.g., anthracene, naphthalene, pyrene and rubrene), acridine dyes (e.g., acridine orange), coumarin dyes (e.g., thiocoumarin), crystal violet, fluorene derivatives (e.g., fluorene and fluorenones), naphthalocyanines, porphyrin derivatives (e.g., copper porphyrin, zinc tetraphenylporphyrin tetrasulfonate, and chlorins such as 5,10,15,20-Tetrakis(m-hydroxyphenyl)chlorin, phthalocyanines, thiazine dyes (e.g., methylene blue and toluidine blue), thioketones, xanthene dyes (e.g., eosin, fluorescein and rose bengal) and the like, as well as mixtures thereof. Other photochemical liquid-state and solid-state sensitizers are also described, respectively, in U.S. Pat. Nos. 5,916,481 and 4,579,837. Those skilled in the art will recognize that sensitizers other than those listed above can be employed, so long as the sensitizer functions adequately as a singlet oxygen-generating agent under the desired conditions of use. Preferably the sensitizer is not toxic or irritating to humans. Sensitizers such as acetonapthone, acetophenone, benzophenone, acridine orange, eosin, fluorene, fluorenone, fluorescein, methylene blue, naphthalocyanines, phthalocyanine, rose bengal, chlorins such as 5,10,15,20-Tetrakis(m-hydroxyphenyl)chlorin, thiocoumarin, toluidine blue and zinc tetraphenylporphyrin tetrasulfonate are especially preferred. The antimicrobial composition preferably contains at least about 0.1 ppm of a photochemical singlet oxygen-generating agent, more preferably about 0.1 to about 50 ppm.

The above-mentioned reaction (1) requires light. A variety of light sources can be employed. The light source is preferably sunlight or room light such as incandescent or fluorescent light. Coherent or narrow spectrum sources such as lasers and light emitting diodes, flash tubes, arc lamps, mercury vapor lamps, sodium lamps and other sources that will be familiar to those skilled in the art can also be employed, so long as the chosen light source provides the desired degree of singlet oxygen generation and safety under the desired conditions of use. Preferably the light source provides both UV and visible energy. For many applications, the light from a typical 60W to 100W incandescent bulb, placed within a few centimeters of the desired target area, will provide sufficient illumination. The intensity of illumination can vary within wide limits, depending in part on the type and concentration of sensitizer. The exposure time can also vary, with exposure times of a few minutes up to a few hours being preferred and exposure times of a few minutes to about one hour being more preferred. As those skilled in the art will appreciate, use of lower intensity illumination or a greater distance between the light source and the antimicrobial composition will typically require a longer exposure time.

The above-mentioned reaction (2) requires the presence or addition of molecular oxygen. Molecular oxygen can be provided in a variety of ways, e.g., from air near the antimicrobial composition, from air or oxygen that is bubbled into the antimicrobial composition, or from air or oxygen already present in the antimicrobial composition or in nearby water. Relatively small amounts of oxygen can be employed. For example, the amount of dissolved oxygen that typically is present in aerated water (about 10⁻⁴ M) will usually suffice for an aqueous antimicrobial composition. Nonaqueous aerated organic solvent solutions often contain even higher levels of dissolved oxygen (e.g., about 10⁻³ M or more for typical organic solvents).

Under the right photochemical singlet oxygen generation conditions, singlet oxygen quantum yields (Φ(¹O₂)=Φ_{T}×Φ_{EN}), where Φ_{T}=triplet quantum yield and Φ_{EN}=energy-transfer yield) as high as 100% can be obtained. For example, in organic solvents and in sodium dodecyl sulfate (“SDS”)/water dispersions containing naphthalene, the reaction

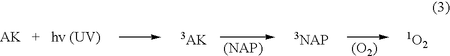

where AK is an aromatic ketone such as benzophenone or acetophenone and NAP is naphthalene proceeds very efficiently with a Φ(¹O₂) of 100%.

Singlet oxygen can be generated in a variety of other ways. For example, gaseous singlet oxygen can be directly photogenerated from ground-state oxygen by laser irradiation, as described in Evans, D. F.; Tucker, J. N. J. Chem. Soc., Faraday Trans. 2 1976, 72, 1661 (oxygen irradiation using a He/Ne laser in a high-pressure cell at 130 atmospheres pressure). Strictly speaking, this method does not involve a discrete singlet oxygen-generating agent. Accordingly, it would not require addition to the antimicrobial composition of a singlet oxygen-generating agent. Instead, singlet oxygen would be generated using the above-described generator and added to a lubricant in situ in order to carry out the method of the invention. Laser irradiation of ground-state oxygen will avoid the need for use of a sensitizer, and consequently will avoid problems associated with the relatively short lifetime of many sensitizers in their excited states. It should be noted in this regard that the lifetime τ (the time required for the concentration to decrease to 1/e where e is the base of the natural logarithm ln) of singlet oxygen in the upper atmosphere is about 64 min, which is much longer than its lifetime in the aqueous or non-aqueous condensed phase.

Various chemical reactions can be employed to generate singlet oxygen. For example, as described in U.S. Pat. No. 3,980,762, a calcined mixture containing lithium, tin, phosphorus and oxygen can be contacted with oxygen at an elevated temperature. In this method, the calcined mixture serves as the singlet oxygen-generating agent. Typically, the calcined mixture would be kept separate from the lubricant, and singlet oxygen would be generated as required, and added to a lubricant as needed.

As described in Foote, C. S.; Wexler, S. JACS 1964, 86, 3879, singlet oxygen can be generated using the reaction:

H₂O₂+XO⁻ (4)

where X is Cl, Br or I. For example, in aqueous solution the reaction NaClO+H₂O₂→NaCl+H₂O+¹O₂ can give Φ(¹O₂) as high as 80%, see Murray, R. W. Chemical Sources of Singlet Oxygen in chapter 3 of Singlet Oxygen, Wassermann, H. H.; Murray, R. W., eds. Academic Press (New York, 1979). However, the reactivity of the hypohalite can interfere with that of singlet oxygen.

Gaseous singlet oxygen can also be generated by the reaction of chlorine gas and a basic hydrogen peroxide solution, as described (for use in an iodine laser) by McDermott, W. E. et al., Appl. Phys. Lett. 32, 469 (1978) and in U.S. Pat. Nos. 4,558,451 and 5,417,928.

Singlet oxygen can also be generated via condensation of halogens such as chlorine or bromine on the surface of a rotating Dewar flask, followed by reaction with basic hydrogen peroxide solution (90% H₂O₂, 15% NaOH), as described in Lee, P. H. and Slafer, W. D., “Chemical Generation of Gaseous Excited Oxygen”, Final Report No. AD A044 024 (August 1977), abstracted at: http://www.cpia.jhu.edu/groups/home/Database/files/database/780368.html.

Singlet oxygen can also be generated via formation of diperoxomolybdate (MoO₆²⁻), using the reaction:

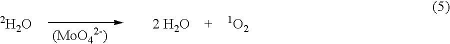

This is a relatively mild chemical generator, and is efficient in water and water/SDS/butanol/CH₂Cl₂ microemulsions of various concentration ratios. For example, using a 2.2/6.5/12.9/78.4% microemulsion containing 5 mmol/kg sodium molybdate dihydrate, and treating 50 g of the microemulsion with 60 μL of 50% H₂O₂, singlet oxygen having a lifetime τ of 45 μs was obtained, see Aubry, J. M. and Bouttemy, S. JACS 1997, 119, 5286.

Singlet oxygen can also be generated from naphthalene endoperoxide singlet oxygen carriers, using the reaction:

1-R, 4-R′ NAP-O₂→1-R, 4-R′ NAP+¹O₂ (30-50° C.) (6)

wherein:

NAP is a water-soluble naphthalene;

NAP-O₂ is a naphthalene endoperoxide substituted at the 1 position with R and at the 4 position with R′;

R is CH₃, (CH₂)₂COONa, or (CH₂)₂COOH; and

R′ is (CH₂)₂COONa or (CH₂)₂COOH,

as described in Aubry, J. M., Cazin, B. and Duprat, F., J. Org. Chem., 54, 726 (1989) and in U.S. Pat. No. 4,436,715. Naphthalene endoperoxides can be prepared by photooxygenation or by the H₂O₂/MoO₄²⁻ reaction (5) shown above.

Singlet oxygen can also be generated using an electric discharge method, such as by excitation of gaseous oxygen using a high-power microwave source, see Ogryzlo, E. A., Gaseous Singlet Oxygen in chapter 2 of Singlet Oxygen, ibid, and in U.S. Pat. Nos. 4,640,782 and 4,095,115. Like the laser irradiation of ground-state oxygen discussed above, this method would not involve a discrete singlet oxygen-generating agent.

Singlet oxygen can also be generated using enzymatic methods, such as reactions employing lactoperoxidase (see Kanofsky, J. R. J. Biol. Chem., 258, 5991 (1983) and Kanofsky, J. R., J. Photochem., 25, 105 (1984)) or haloperoxidase (see Khan, A. U., JACS, 105, 7195 (1983), Khan, A. U., J. Photochem., 5, 327 (1984) and U.S. Pat. No. 6,033,662).

The compositions of the invention also contain at least one lubricant. As used in this invention, the term “lubricant” refers to a liquid or solid material (other than water) that reduces friction between two sliding surfaces. A variety of lubricants can be employed, including natural lubricants, petroleum lubricants, synthetic oils and greases. Preferred natural lubricants include vegetable oils, fatty oils, animal fats, and other materials that can be obtained from natural sources such as seeds, plants, fruits, or animal tissue. Preferred petroleum lubricants include mineral oils, petroleum distillates, and petroleum products. Preferred synthetic oils include synthetic hydrocarbons, organic esters, poly(alkylene) glycols, high molecular weight alcohols, carboxylic acids, phosphate esters, perfluoroalkylpolyethers (“PFPEs”), silicates, silicones such as silicone surfactants, chlorotrifluoroethylene, polyphenyl ethers, polyethylene glycols, oxypolyethylene glycols, copolymers of ethylene and propylene oxide, and the like. Preferred solid lubricants include molybdenum disulfide, boron nitride, graphite, inorganic particles (such as the silica and alumina particles described in copending U.S. patent application Ser. No. 09/604,469, filed Jun. 27, 2000, the disclosure of which is incorporated herein by reference), silicone gums and particles, polytetrafluoroethylene (“PTFE”) particles, fluoroethylene-propylene (“FEP”) copolymers, perfluoroalkoxy (“PFA”) resins, ethylene-chloro-trifluoroethylene (“ECTFE”) alternating copolymers, poly(vinylidene fluoride) (“PVDF”), waxes, fatty acids, phosphate esters and mixtures thereof. The lubricant can be water-soluble, water-dispersible or water-insoluble. Useful water-soluble or dispersible lubricants include, but are not limited to, polymers of one or more of ethylene oxide, propylene oxide, methoxy polyethylene glycol, or an oxyethylene alcohol. Other lubricants will be familiar to those skilled in the art, and include the lubricants described, for example, in U.S. Pat. Nos. 4,828,727, 4,944,889, 5,009,801, 5,062,979, 5,073,280, 5,174,914, 5,334,322, 5,352,376, 5,474,692, 5,559,087, 5,565,127, 5,663,131, 5,688,747, 5,672,401, 5,747,430 and 5,935,914, and in published PCT Application Nos. WO 98/56881 and WO 01/07544 A1.

Preferred commercially available lubricants include aqueous lubricants and lubricant concentrates available under the LUBODRIVE, KX-5120 and MICROGLIDE trademarks from Ecolab; aqueous lubricants and lubricant concentrates available under the DICOLUBE or STAR TRACK trademarks from Diversey Lever such as DICOLUBE™ PL, TPB and STAR TRACK™ VL15; aqueous lubricants and lubricant concentrates available under the AFCO LUBE trademarks from Fergusson, Inc; aqueous lubricants and lubricant concentrates available under the WEST GLIDE and SUPER WEST GLIDE trademarks from West Agro Corporation; oleic acid, corn oil, mineral oils available under the BACCHUS trademark from Vulcan Oil and Chemical Products; fluorinated oils and fluorinated greases available under the KRYTOX trademark from DuPont Chemicals; siloxane fluids such as SF96-5 and SF 1147 available from GE Silicones; synthetic oils and mixtures thereof with PTFE available under the trademark SUPER LUBE from Synco Chemical; polyalkylene glycols such as UCON™ LB625 and CARBOWAX™ materials from Union Carbide Corp.; the EVERGLIDE™ and ULTRAGLIDE™ series of micronized wax powders, dispersions and emulsions such as EVERGLIDE UV-636 (25% carnauba wax emulsified in tripropylene glycol diacrylate), EVERGLIDE UV-231 D (35% fluoroethylene wax dispersed in tripropylene glycol diacrylate), ULTRAGLIDE UV-701 (40% PTFE dispersed in tripropylene glycol diacrylate) and ULTRAGLIDE UV-801 (35% PTFE in tridecyl stearate), available from Shamrock Technologies, Inc.; high performance PTFE lubricant products such as NANOFLON™ M020, FLUOROSLIP™ 225 and NEPTUNE™ 5031 also available from Shamrock Technologies Inc.; and the MICROSPERSION™, POLYFLUO™ AND SYNFLUO™ series of micronized waxes such as MICROSPERSION 190-50 50% aqueous dispersion of polyethylene wax and PTFE and POLYFLUO 190 micronized fluorocarbon available from Micro Powders Inc.

The lubricant can include a hydrophilic material whose lubricating properties are enhanced by contact with a polar liquid, as described in copending U.S. patent application Ser. No. 09/735,814, filed Dec. 13, 2000, the disclosure of which is incorporated herein by reference. Preferably, such lubricating properties are enhanced even after contact with the polar liquid ceases. Preferably, the lubricant is part of a coating that is hardened, and that becomes swollen by or will absorb the polar liquid. A variety of polar liquids can be employed, including water, alcohols such as isopropyl alcohol, polyols such as glycerol and polyethylene glycols, and mixtures thereof. Water is a preferred polar liquid. Following contact of the coating by the polar liquid, the coating will have a reduced coefficient of friction or “COF”. A preferred reduced COF is less than about 0.14, more preferably less than about 0.1. The coating preferably is relatively durable and following cure will remain in place on a lubricated sliding surface. However, if desired the coating can be formulated so that it can be removed using a suitable agent, e.g., a solvent, heat such as steam heat, aqueous detergent solution, or other removal technique.

The lubricant can contain a mixture of a water-miscible silicone material and a water-miscible lubricant as described in copending U.S. patent application Ser. No. 09/596,599, filed Jun. 16, 2000, the disclosure of which is incorporated herein by reference. The silicone material and hydrophilic lubricant are sufficiently water-soluble or water-dispersible so that when added to water at the desired use level they form a stable solution, emulsion or suspension.

The lubricant can include a phase-separating mixture of a hydrophilic lubricating material and an oleophilic lubricating material whose specific gravity is less than or equal to the specific gravity of the hydrophilic lubricating material, as described in U.S. Pat. No. 6,207,622. Prior to application to a sliding surface, the mixture is agitated or otherwise maintained in a mixed but unstable state. Following application, the hydrophilic lubricating material and oleophilic lubricating material tend to undergo phase-separation, and the oleophilic lubricating material tends to form a continuous or discontinuous film atop the hydrophilic lubricating material thereby providing a water-repelling lubricating layer having reduced water sensitivity.

The antimicrobial composition can be a liquid, semi-solid or solid at the time of application to a sliding surface. Preferably, the antimicrobial composition is a liquid having a viscosity that will permit it to be pumped and readily applied to a desired sliding surface or surfaces (e.g., a beverage conveyor or containers), and that will facilitate rapid film formation whether or not the sliding surfaces are in motion with respect to one another. The antimicrobial composition can be formulated so that it exhibits shear thinning or other pseudo-plastic behavior, manifested by a higher viscosity (e.g., non-dripping behavior) when at rest, and a much lower viscosity when subjected to shear stresses such as those provided by pumping, spraying or brushing the antimicrobial composition. This behavior can be brought about by, for example, including appropriate types and amounts of thixotropic fillers (e.g., treated or untreated fumed silicas) or other rheology modifiers in the antimicrobial composition. The antimicrobial composition can be applied to a sliding surface in a constant or intermittent fashion. Preferably, the antimicrobial composition is applied in an intermittent fashion in order to minimize the amount of applied lubricant. For example, in beverage conveyor applications, the antimicrobial composition can be applied to the conveyor for a period of time during which at least one complete revolution of the conveyor takes place. Application of the antimicrobial composition can then be halted for a period of time (e.g., minutes or hours) and then resumed for a further period of time (e.g., one or more further conveyor revolutions). The lubricant coating should be sufficiently thick to provide the desired degree of lubrication, and sufficiently thin to permit economical operation and to discourage drip formation. For beverage conveyor applications, the lubricant coating thickness preferably is maintained at at least about 0.0001 mm, more preferably about 0.001 to about 2 mm, and most preferably about 0.005 to about 0.5 mm.

As mentioned above, the lubricant can be, inter alia, an aqueous or non-aqueous liquid lubricant. Aqueous lubricants typically will be formulated to contain or to be diluted with significant amounts of water, e.g., at water:lubricant dilution ratios of about 100:1 to 500:1. Non-aqueous lubricants typically will be formulated to contain lower amounts of solvent or carrier, e.g., as neat solutions containing no solvent or carrier or as relatively concentrated solutions or dispersions. Antimicrobial compositions containing such non-aqueous lubricants can be applied relatively sparingly as thin, dry lubricating films. Thus in contrast to antimicrobial compositions containing aqueous lubricants, non-aqueous antimicrobial compositions can provide dry lubrication of conveyors and containers, a cleaner and drier conveyor line and working area, and reduced lubricant usage, thereby reducing waste, cleanup and disposal problems.

As mentioned above, the lubricant can be, inter alia, a solid lubricant. If desired, the solid can be dispersed or suspended in an aqueous or non-aqueous liquid. A preferred amount of solid lubricant is at least about 1 wt. %, more preferably about 3 to about 50 wt. %, and most preferably about 5 to about 30 wt. %, based on the total weight of the antimicrobial composition.

The antimicrobial compositions of the invention optionally can contain at least one chelating agent. The presence of a chelating agent can synergistically increase the level of antimicrobial activity. A variety of chelating agents can be employed, including tris(hydroxymethyl)aminomethane (“TRIS”), amino carboxylates (e.g., amino succinates or acetates such as ethylenediamine disuccinate, ethylenediamine tetraacetate (“EDTA”), N-hydroxyethylethylenediamine triacetate; ethylenediamine tetraproprionates; triethylenetetraamine hexacetate; or diethylenetriamine pentaacetate); nitrilotriacetates; ethanoldiglycines; amino phosphonates; polysubstituted aromatic chelating agents; other chelating agents that will be familiar to those skilled in the art; as well as mixtures thereof or salts thereof (e.g., alkali metal, ammonium, or substituted ammonium salts). Preferably, the antimicrobial composition contains about 0.1 to about 1000 ppm, more preferably about 1 to about 100 ppm chelating agent based on the total weight of the antimicrobial composition. A 10:1 molar mixture of EDTA and TRIS (e.g., at a concentration of about 10 ppm) is especially preferred.

The antimicrobial compositions of the invention optionally can contain at least one surfactant. The presence of a surfactant can also synergistically increase the level of antimicrobial activity. A variety of surfactants can be employed, including anionic, cationic, nonionic, amphoteric or zwitterionic surfactants such as alkyl benzene sulfonates, alkyl sulfates, unsaturated sulfates, alkyl alkoxy sulfates, alkyl alkoxy carboxylates, glycerol ethers, alkyl polyglycosides and their corresponding sulfated polyglycosides, alpha-sulfonated fatty acid esters, alkyl ethoxylates, alkyl phenol alkoxylates, betaines, sulfobetaines, N-alkyl polyhydroxy fatty acid amides, N-alkoxy polyhydroxy fatty acid amides, other surfactants that will be familiar to those skilled in the art, and mixtures thereof. Preferably the antimicrobial composition contains about 1 ppm to about 10 wt. %, more preferably about 10 ppm to about 1 wt. % surfactant based on the total weight of the antimicrobial composition. A composition containing about 0.1 wt. % anionic or nonionic surfactant is especially preferred.

Those skilled in the art will appreciate that the antimicrobial composition can include a variety of optional adjuvants. For example, the antimicrobial compositions can contain additional antimicrobial agents (e.g., carboxylic acids, diacids, or triacids such as butyric acid, heptanoic acid, octanoic acid, nonanoic acid, decanoic acid, salycic acid, mandelic acid, succinic acid, adipic acid, glutaric acid, EDTA and citric acid), carboxylic esters (e.g., p-hydroxy alkyl benzoates and alkyl cinnamates), sulfonic acids (e.g., dodecylbenzene sulfonic acid), iodo-compounds or active halogen compounds (e.g., iodine, interhalides, polyhalides, metal hypochlorites, hypochlorous acid, metal hypbromites, hypobromous acid, chloro- and bromo-hydantoins, chlorine dioxide and sodium chlorite), isolated or equilibrium derived or isolated peracids such as chloroperbenzoic acids, peracetic acid, perheptanoic acid, peroctanoic acid, perdecanoic acid, performic acid, percitric acid, perglycolic acid, perlactic acid, perbenzoic acid, and monoester peracids derived from diacids or diesters (e.g., such as adipic, succinic, glutaric or malonic acid and mixtures thereof), organic peroxides including benzoyl peroxide, alkyl benzoyl peroxides, and mixtures thereof, phenolic derivatives (e.g., o-phenyl phenol, o-benzyl-p-chlorophenol, tert-amyl phenol and C₁-C₆ alkyl hydroxy benzoates), quaternary ammonium compounds (e.g., alkyldimethylbenzyl ammonium chloride or dialkyldimethyl ammonium chloride), other antimicrobial agents that will be familiar to those skilled in the art, and mixtures thereof. Other adjuvants include electrolytes, colorants, foam inhibitors or foam generators, plasticizers, adhesion promoters, cracking inhibitors (such as PET stress cracking inhibitors), viscosity modifiers, solvents, coating aids and antistatic agents. The amounts and types of such adjuvants will be apparent to those skilled in the art.

For applications involving plastic containers, care should be taken to avoid the use of components or contaminants that might promote environmental stress cracking of the container. Preferably, antimicrobial compositions for use in such applications have a total alkalinity equivalent to less than about 100 ppm CaCO₃, more preferably less than about 50 ppm CaCO₃, and most preferably less than about 30 ppm CaCO₃, as measured in accordance with Standard Methods for the Examination of Water and Wastewater, 18th Edition, Section 2320, Alkalinity.

A variety of sliding surfaces can be antimicrobially lubricated with the antimicrobial compositions of the invention. The invention is particularly well suited to coating conveyors and containers, e.g. beverage conveyors and containers. Other representative sliding surfaces include those found in food processing equipment, pharmaceutical manufacturing equipment, medical instruments and other equipment having surfaces for which control of microbial levels is required or desired for health and safety reasons. In beverage conveyor applications, conveyor parts that support, guide or move the containers preferably are coated with the antimicrobial composition. Such parts include belts, chains, gates, chutes, sensors, and ramps having surfaces made of fabrics, metals, plastics, composites, or combinations of these materials. Sensors can be used at one or more points along the conveyor line to determine when the antimicrobial composition may have become sufficiently depleted to require reapplication.

The antimicrobial composition can be applied to a wide variety of containers including beverage containers; food containers; household or commercial cleaning product containers; and containers for oils, antifreeze or other industrial fluids. The containers can be made of a wide variety of materials including glasses; plastics (e.g., polyolefins such as polyethylene and polypropylene; polystyrenes; polyesters such as polyethylene terephthalate (“PET”) and polyethylene naphthalate (“PEN”); polyamides, polycarbonates; and mixtures or copolymers thereof); metals (e.g., aluminum, tin or steel); papers (e.g., untreated, treated, waxed or other coated papers); ceramics; and laminates or composites of two or more of these materials (e.g., laminates of PET, PEN or mixtures thereof with another plastic material). The containers can have a variety of sizes and forms, including cartons (e.g., waxed cartons or TETRAPACK™ boxes), cans, bottles and the like. Although any desired portion of the container can be coated with the antimicrobial composition, the antimicrobial composition preferably is applied only to parts of the container that will come into contact with the conveyor or with other containers. Preferably, the antimicrobial composition is not applied to portions of thermoplastic containers that are prone to stress cracking. In a preferred embodiment of the invention, the antimicrobial composition is applied to the crystalline foot portion of a blow-molded, footed PET container (or to one or more portions of a conveyor that will contact such foot portion) without applying significant quantities of antimicrobial composition to the amorphous center base portion of the container. Also, the antimicrobial composition preferably is not applied to portions of a container that might later be gripped by a user holding the container, or, if so applied, is preferably removed from such portion prior to shipment and sale of the container. For some such applications the antimicrobial composition preferably is applied to the conveyor rather than to the container, in order to limit the extent to which the container might later become slippery in actual use.

Application of the antimicrobial composition can be carried out using any suitable technique including spraying, wiping, brushing, drip coating, roll coating, and other methods for application of a thin film. If desired, the antimicrobial composition can be applied using spray equipment designed for the application of conventional aqueous conveyor lubricants, modified as need be to suit the rheological characteristics of the antimicrobial compositions of the invention.

If desired, the antimicrobial composition can be cured or hardened following application to a surface, using thermal- or radiation-induced curing as described in copending U.S. patent application Ser. No. 09/604,469, filed Jun. 27, 2000, the disclosure of which is incorporated herein by reference. The resulting coating is dry to the touch following cure, and relatively water-insoluble (that is, the cured coating will not be washed away when exposed to water). The coating can be reapplied as needed, (e.g., on a conveyor line during operation thereof), to compensate for coating wear. An entire lubricated article (e.g., a container or conveyor) can be coated or treated, but it is usually preferred to coat only those surfaces of the lubricated article or articles that will come into sliding contact with one another. The antimicrobial composition preferably is substantially non-dripping prior to cure; that is, preferably, the majority of the composition remains on the lubricated article following application until such time as the antimicrobial composition is cured.

When the antimicrobial composition contains a photochemical singlet oxygen-generating agent and is curable or hardenable, care should be taken to ensure that the use of a thermal or radiation care system to harden the antimicrobial composition does not interfere with the desired mechanism for photochemical generation of singlet oxygen. With that caveat in mind, a thermally stable antimicrobial composition can be cured using a variety of energy sources that will generate sufficient heat to initiate and promote hardening of the antimicrobial composition. Suitable sources include conventional heaters, infrared radiation sources, and microwave energy sources. With the same caveat in mind, a radiation curable antimicrobial composition can be cured using a variety of energy sources that will induce a photochemical reaction and thereby harden the composition, including ultraviolet radiation, visible light, infrared radiation, X-rays, gamma rays, and electron beams. Preferred energy sources include mercury vapor arc lamps, fluorescent lamps, tungsten halide lamps, visible lasers and infrared lamps. A UV-cured antimicrobial composition coating can be obtained on the bottom of a container by passing the container through a coating station to apply the antimicrobial composition to the bottom of the container and photoexposing the antimicrobial composition solution to cure it into a hardened film. Photoexposure can be carried out from underneath the container by transmitting the curing energy through the conveyor belt. In such a case, the conveyor belt should be sufficiently transparent to the desired wavelength of curing energy so that efficient cure will take place. Also, the coated container can be photoexposed from above or from one or more sides of the container. In such a case, the container should be sufficiently transparent to the desired wavelength of curing energy so that efficient cure will take place.

The invention is further illustrated in the following non-limiting examples, in which all parts and percentages are by weight unless otherwise indicated.

### EXAMPLE 1

### Light Activated Antimicrobial Beverage Lubricant

A commercial aqueous lubricant (MICROGLIDE™ beverage container lubricant, Ecolab Inc.) was modified to remove the antimicrobial additive that ordinarily is present in this lubricant's formulation, then diluted to 0.5% in water and combined with varying amounts of toluidine blue photo-activator dye. The resulting antimicrobial compositions were exposed to a variety of lighting conditions and evaluated for antimicrobial activity at room temperature (about 22° to 26° C.) against S. aureus and P. aeruginosa. For lighting exposures involving a 90W lamp, the temperature tended to be somewhat higher, but remained below 41° C. By evaluating a control composition with a light source but without a dye, the effect of heating caused by the light source could be assessed. The results are set out below in Table 1.

TABLE 1
The Antimicrobial Effect of a Photo-Activator in an Aqueous Lubricant

| | | | | | Microbial | Microbial |
|---|---|---|---|---|---|---|
| | | | | | Log | Log |
| | | Dye | | | Reduction | Reduction |
| Run | | Amount | Light | | (30 | (60 |
| No. | Dye | (in Lubricant) | Source | Organism | Minutes) | Minutes) |
| 1-1 | None | 0 ppm | 90 W | S. aureus | — | <0.8 |
| (Control) | | | Lamp¹ | P. aeruginosa | — | — |
| 1-2 | Toluidine | 3.0 ppm | None² | S. aureus | 0.1 | 0.1 |
| (Control) | Blue | | | P. aeruginosa | <0.6 | <0.6 |
| 1-3 | Toluidine | 3.0 ppm | Room | S. aureus | 1.2 | 1.5 |
| | Blue | | Light³ | P. aeruginosa | 1.5 | 2.2 |
| 1-4 | Toluidine | 3.0 ppm | 90 W | S. aureus | 2.8 | 3.1 |
| | Blue | | Lamp | P. aeruginosa | >7.1 | >7.1 |
| 1-5 | Toluidine | 1.5 ppm | Room | S. aureus | 1.1 | 1.5 |
| | Blue | | Light | P. aeruginosa | 1.5 | >7.1 |
| 1-6 | Toluidine | 1.7 ppm | 90 W | S. aureus | 1.7 | 2.5 |
| | Blue | | Lamp | P. aeruginosa | >7.1 | >7.1 |
| 1-7 | Toluidine | 1.5 ppm | 90 W | S. aureus | 1.7 | 2.5 |
| | Blue | | Lamp | P. aeruginosa | >7.1 | >7.1 |
| 1-8 | Toluidine | 1.0 ppm | 90 W | P. aeruginosa | >5.8 | — |
| | Blue | | Lamp | | | |

1. 90 W lamp at about 30.5 cm from the test solution.

2. The experiment was done in the dark without light impingement.

3. Room light using laboratory broad-spectrum fluorescent lighting located approximately 1.8 meters from the test solution.

Comparison of the control experiments (Run Nos. 1-1 and 1-2) to the antimicrobial compositions of the invention (Run Nos. 1-3 through 1-8) shows that microbial reduction improved significantly (amounting in some cases to several orders of magnitude) when toluidine blue was present and the composition was exposed to light. Antimicrobial efficacy improved as light intensity increased.

### EXAMPLE 2

### Light Activated Oxidizing Antimicrobial Chain Lubricant

Using the method of Example 1, a commercial beverage container lubricant (KX-5120™ lubricant, Ecolab Inc.) was diluted to 0.5% in water and combined with varying amounts of toluidine blue photo-activator dye and hydrogen peroxide. The resulting antimicrobial compositions were exposed to a variety of lighting conditions and evaluated for antimicrobial activity against S. aureus and E. coli. The results are set out below in Table 2.

TABLE 2
The Antimicrobial Effect of a Photo-Activator in
an Aqueous Peroxide-Based Lubricant

| | | | | | Microbial | Microbial |
|---|---|---|---|---|---|---|
| | | | | | Log | Log |
| | | Dye/H₂O₂ | Light | | Reduction | Reduction |
| Run No. | Dye | Amount | Intensity | Organism | (30 Minute) | (60 Minute) |
| 2-1 | Toluidine | 3.0 ppm/100 | None | S. aureus | 0.1 | 0.1 |
| (Control) | Blue | ppm | | E. coli | — | 0.4 |
| 2-2 | None | 0 ppm/0 | Room | S. aureus | 0.04 | <0.1 |
| (Control) | | ppm | Light | E. coli | 0.3 | 0.3 |
| 2-3 | None | 0 ppm/100 | Room | S. aureus | 0.8 | 0.8 |
| (Control) | | ppm | Light | E. coli | 0.1 | 0.2 |
| 2-4 | None | 0 ppm/0 | 90 W | S. aureus | 0.2 | 0.3 |
| (Control) | | ppm | | E. coli | 0.2 | 1.2 |
| 2-5 | None | 0 ppm/100 | 90 W | S. aureus | 1.5 | 2.4 |
| (Control) | | ppm | | E. coli | 0.3 | 0.5 |
| 2-6 | Toluidine | 3.0 ppm/0 | Room | S. aureus | 4.0 | 5.2 |
| | Blue | ppm | Light | E. coli | >7.1 | >7.1 |
| 2-7 | Toluidine | 3.0 ppm/100 | Room | S. aureus | 4.8 | 5.6 |
| | Blue | ppm | Light | E. coli | 4.4 | >7.1 |
| 2-8 | Toluidine | 3.0 ppm/0 | 90 W | S. aureus | >6.9 | >6.9 |
| | Blue | ppm | | E. coli | >7.1 | >7.1 |
| 2-9 | Toluidine | 3.0 ppm/100 | 90 W | S. aureus | >6.9 | >6.9 |
| | Blue | ppm | | E. Coli | >7.1 | >7.1 |

Comparison of the control experiments (Run Nos. 2-1 through 2-5) to the antimicrobial compositions of the invention (Run Nos. 2-6 through 2-9) shows that microbial reduction improved significantly (amounting in some cases to several orders of magnitude) when toluidine blue was present and the antimicrobial composition was exposed to light. Antimicrobial efficacy improved as light intensity increased, and under low light intensity when peroxide was present.

### EXAMPLE 3

### Light Activated Chain Lubricant

Using the method of Example 1, a chain lubricant containing 56.8% glycerin (96% USP), 41.7% deionized water and 1.5% polydimethyl siloxane was combined with varying amounts of toluidine blue dye. The resulting antimicrobial compositions were exposed to a variety of lighting conditions and evaluated for antimicrobial activity against S. aureus and P. aeruginosa. The results are set out below in Table 3.

TABLE 3
The Antimicrobial Effect of a Photo-Activator in a Chain Lubricant

| | | | | | Microbial | Microbial |
|---|---|---|---|---|---|---|
| | | | | | Log | Log |
| | | | | | Reduction | Reduction |
| | | Dye | Light | | (30 | (60 |
| Run No. | Dye | Amount | Source | Organism | Minute) | Minute) |
| 3-1 | None | 0 ppm | Room | S. aureus | — | 0.1 |
| (Control) | | | Light | P. | — | 0.3 |
| | | | | aeruginosa | | |
| 3-2 | Toluidine | 3.0 ppm | None | S. aureus | — | None |
| (Control) | Blue | | | P. | — | 0.2 |
| | | | | aeruginosa | | |
| 3-3 | Toluidine | 1.5 ppm | Room | S. aureus | 0.4 | 0.8 |
| | Blue | | Light | P. | 0.5 | 0.7 |
| | | | | aeruginosa | | |
| 3-4 | Toluidine | 3.0 ppm | Room | S. aureus | 1.2 | 2.2 |
| | Blue | | Light | P. | 0.7 | 0.8 |
| | | | | aeruginosa | | |

Comparison of the results of the control experiments (Run Nos. 3-1 and 3-2) to the antimicrobial compositions of the invention (Run Nos. 3-3 and 3-4) shows the antimicrobial effectiveness of the dye in a non-aqueous system.

### EXAMPLE 4

### Evaluation of Additional Photo-Activator Dyes

A variety of photo-activator dyes were dissolved in sterile water from a MILLI-Q™ Ultrapure Water System (Millipore Corp.) at varying concentrations, optionally exposed to light for 30 or 60 minutes using a 90W lamp located about 30 cm from the test solution and evaluated for antimicrobial activity against one or more of E. coli, S. aureus, S. typhi and P. aeruginosa. The results are set out below in Table 4.

TABLE 4
The Antimicrobial Effect of Various Photo-Activator Dyes
in Aqueous Suspensions

| | | | | Microbial | Microbial |
|---|---|---|---|---|---|
| | | | | Log | Log |
| | | Dye | | Reduction | Reduction |
| Run No. | Dye | Amount | Organism | (30 Minute) | (60 Minute) |
| 4-1 | Toluidine | 3.1 ppm | S. aureus | 0.1 | 0.1 |
| (Control | Blue | | P. aeruginosa | <0.6 | <0.6 |
| w/no | | | | | |
| light) | | | | | |
| 4-2 | Acridine | 6.3 ppm | E. coli | — | 6.8 |
| | Orange | | S. aureus | 4.7 | >6.4 |
| | | | S. typhi | — | 5.8 |
| | | | P. aeruginosa | 5.2 | 5.0 |
| 4-3 | Rose | 10 ppm | E. coli | — | 0.5 |
| | Bengal | | S. aureus | 3.0 | >6.4 |
| | | | S. typhi | — | 2.0 |
| | | | P. aeruginosa | 0.4 | 0.7 |
| 4-4 | Crystal | 10 drops/ | E. coli | — | 0.0 |
| | Violet | 800 ml | S. aureus | — | — |
| 4-5 | Toluidine | 3.1 ppm | E. coli | 2.8 | 5.4 |
| | Blue | | | | |
| 4-6 | Toluidine | 5 ppm | E. coli | — | 6.1 |
| | Blue | | S. aureus | — | 5.4 |
| 4-7 | Methylene | 4.5 ppm | E. coli | — | 6.0 |
| | Blue | | S. aureus | 4.3 | >6.4 |
| | | | S. typhi | — | >6.6 |
| | | | P. aeruginosa | 0.7 | 1.6 |
| 4-8 | Eosin B | 5 ppm | E. coli | — | 0.0 |
| 4-9 | 50/50 | 16.5 ppm | E. coli | — | 1.0 |
| | mixture of | | S. aureus | 6.1 | 6.4 |
| | Rose | | S. typhi | — | 0.5 |
| | Bengal/ | | P. aeruginosa | 0.3 | 0.5 |
| | Acridine | | | | |
| | Orange | | | | |

The results in Table 4 show a variety of singlet oxygen-generating agents and their effectiveness against a variety of microorganisms. In the absence of light, little or no microbial log reduction was or would be obtained.

### EXAMPLE 5

### Light Activated Antimicrobial Chain Lubricants

Using the method of Example 1, a commercial beverage container lubricant (MICROGLIDE™ lubricant, Ecolab Inc.) was diluted to 0.1% or 0.5% in water and combined with varying amounts of toluidine blue photo-activator dye. The resulting antimicrobial compositions were exposed for 30 and 60 minutes using a 90W lamp located about 30.5 cm from the test surface and evaluated for antimicrobial activity against S. aureus and P. aeruginosa. The results are set out below in Table 5.

TABLE 5
The Antimicrobial Effect of Various Photo-Activator Dyes
in an Aqueous Lubricant

| | | | | | Microbial | Microbial |
|---|---|---|---|---|---|---|
| | | | | | Log | Log |
| | | Dye | Lube | | Reduction | Reduction |
| Run No. | Dye | Amount | Amount | Organism | (30 Minute) | (60 Minute) |
| 5-1 | Toluidine | 3 ppm | 0.5% | S. aureus | 1.2 | 1.5 |
| | Blue | | | P. aeruginosa | 1.5 | 2.2 |
| 5-2 | Methylene | 3 ppm | 0.1% | S. aureus | 3.6 | >6.3 |
| | Blue | | | P. aeruginosa | >7.0 | >7.0 |
| 5-3 | Rose | 3 ppm | 0.1% | S. aureus | 1.6 | 5.9 |
| | Bengal | | | P. aeruginosa | >7.0 | >7.0 |
| 5-4 | Acridine | 3.5 ppm | 0.5% | S. aureus | 1.2 | 3.4 |
| | Orange | | | P. aeruginosa | >7.3 | >7.3 |
| 5-5 | Acridine | 7 ppm | 0.5% | S. aureus | 1.1 | >7.3 |
| | Orange | | | P. aeruginosa | >7.3 | >7.3 |

The results in Table 5 show the use of a variety of dyes and dye concentrations.

### EXAMPLE 6

### pH Effects

A pH kill rate profile for toluidine blue photo-activator dye was determined in aqueous solution by adding 1.0 ppm of dye to sterile water solutions adjusted to various pH levels, and exposing the resulting antimicrobial compositions for 15, 30 and 60 minutes to light from a 90W lamp located about 30.5 cm from the test surface. The antimicrobial compositions were evaluated for antimicrobial activity against E. coli and S. aureus. The results are set out below in Table 6.

TABLE 6
Antimicrobial pH Profile

| | | | Microbial | Microbial | Microbial |
|---|---|---|---|---|---|
| | | | Log | Log | Log |
| | | | Reduction | Reduction | Reduction |
| Run | Solution | | (15 | (30 | (60 |
| No. | pH | Organism | Minutes) | Minutes) | Minutes) |
| 6-1 | 4 | E. coli | 0.4 | 0.8 | 6.2 |
| | | S. aureus | 0.8 | 2.8 | >8.2 |
| 6-2 | 6 | E. coli | 6.7 | 8.0 | >8.0 |
| | | S. aureus | 6.6 | >8.2 | >8.2 |
| 6-3 | 8 | E. coli | 7.7 | >8.0 | >8.0 |
| | | S. aureus | >8.2 | >8.2 | >8.2 |
| 6-4 | 10 | E. coli | >8.0 | >8.0 | >8.0 |
| | | S. aureus | 4.9 | >8.2 | >8.2 |

The results in Table 6 show that toluidine blue dye worked most rapidly at neutral to alkaline pH levels. However, longer exposure times provided good microbial reduction at all pH levels.

### EXAMPLE 7

### The Effect of Oxygen on Microbial Reduction

The effect of adding oxygen to a photo-activated dye system was evaluated in aqueous solution by adding 1.3 ppm of toluidine blue dye to sterile water, covering the resulting antimicrobial composition with a barrier film, and bubbling nitrogen or oxygen gas through the composition while exposing the antimicrobial composition for 15, 30 and 60 minutes to light from a 90W lamp located about 30.5 cm from the test surface. Exposure to nitrogen gas tended to carry away oxygen above the antimicrobial composition and reduce the amount of dissolved oxygen. Exposure to oxygen gas tended to increase the amount of dissolved oxygen. The antimicrobial compositions were evaluated for antimicrobial activity against E. coli and S. aureus. The results are set out below in Table 7.

TABLE 7
Exposure to Nitrogen or Oxygen Gas

| | | Bubbling/ | Microbial | Microbial |
|---|---|---|---|---|
| | | Exposure | Log | Log |
| | | Time | Reduction, | Reduction, |
| Run No. | Condition | (Minutes) | E. coli | S. aureus |
| 7-1 | Bubbled Nitrogen | 15 | 0.4 | 0.2 |
| | | 30 | 0.5 | 0.3 |
| | | 60 | 1.7 | 2.5 |
| 7-2 | Bubbled Oxygen | 15 | >7.2 | 3.4 |
| | | 30 | >7.2 | 4.8 |
| | | 60 | >7.2 | >6.8 |

The results in Table 7 show the beneficial effect of adding oxygen to the system and the negative effect of its exclusion. The blue dye coloration faded very rapidly when nitrogen was bubbled into the system (caused by a photoreduction reaction that occurs in the absence of oxygen), but the color intensity returned after re-oxidation.

### EXAMPLE 8

### Lubricant Conveyor Test

A photo-activator dye was evaluated during the operation of a beverage conveying line. MICROGLIDE™ beverage container lubricant (Ecolab) was prepared without an additional inline biocidal agent and diluted to 1 wt % using city tap water. 1 ppm of toluidine blue dye was added to the lubricant and the resulting antimicrobial composition was sprayed onto a short-track conveying system equipped with 4 90W lamps hung about 30.5 cm from the test surface. The conveyor speed was about 30 m/min. The lubricant was supplied at a pressure of about 0.14 MPa and the lubricant delivery rate was approximately 7.5 to 11.3 liters/hour. A sample of the antimicrobial composition and a control prepared without the dye were taken after four hours of operation and evaluated for P. aeruginosa. The results are set out below in Table 8.

TABLE 8
The Antimicrobial Effect of a Photo-Activator Dye
on a Beverage Conveyor Line

| | | | | | Microbial |
|---|---|---|---|---|---|
| | | Dye | Lubricant | | Log |
| Run No. | Dye | Amount | Amount | Organism | Reduction |
| 8-1 | None | 0 ppm | 0.1% | P. | <.1 |
| (Control) | | | | aeruginosa | |
| 8-2 | Toluidine | 1 ppm | 0.1% | P. | 4.0 |
| | Blue | | | aeruginosa | |

The results shown in Table 8 illustrate the positive effect of using a singlet oxygen generator during conveyer operation, even at a very low dye addition level.

### EXAMPLE 9

### Antifungal Efficacy

Varying amounts of toluidine blue photo-activator dye were combined with sterile water, a commercial lubricant (MICROGLIDE™ beverage container lubricant, Ecolab Inc.) or a 0.5% solution of Tris-EDTA. The resulting antimicrobial compositions were exposed for 60 to 240 minutes to light from a 90W lamp located about 30.5 cm from the test surface and evaluated for antimicrobial activity against the fungus Aspergillus sp. The results are set out below in Table 9.

TABLE 9
Antifungal Effect

| | | | Exposure | Microbial Log |
|---|---|---|---|---|
| Run | Toluidine | | Time | Reduction, |
| No. | Blue Amount | Test Solution | (Minutes) | Aspergillus sp. |
| 9-1 | 1.0 ppm | Sterile Water | 60 | 0.1 |
| 9-2 | 4.0 ppm | Sterile Water | 60 | 0.3 |
| 9-3 | 4.0 ppm | 0.1 wt % | 60 | 0.6 |
| | | Lubricant in | | |
| | | Tap Water | | |
| 9-4 | 5.6 ppm | Sterile Water | 60 | 0.1 |
| | | | 120 | 0.6 |
| | | | 240 | 3.2 |
| 9-5 | 5.6 ppm | 0.5 wt % Tris- | 60 | 2.6 |
| | | EDTA | | |

The results in Table 9 show that a positive antifungal effect can be achieved under a variety of conditions and that the effect was not harmed by the presence of the lubricant. Addition of tris-EDTA solution gave a synergistic increase in antifungal efficacy.

### EXAMPLE 10

### Chelant Addition

Using the method of Example 9, varying amounts of toluidine blue photo-activator dye were combined with a 0.5% solution of tris-EDTA in water. The resulting compositions were exposed for 30 minutes to light from a 90W lamp located about 30.5 cm from the test surface and evaluated for antimicrobial activity against S. aureus and P. aeruginosa. The results are set out below in Table 10.

TABLE 10
Antimicrobial Improvement Using a Chelant

| | | | | Microbial |
|---|---|---|---|---|
| | Toluidine | | | Log |
| Run | Blue | | | Reduction |
| No. | Amount | Test Solution | Organism | (30 Minute) |
| 10-1 | 0.0 ppm | 0.5 wt % Tris- | S. aureus | 0.0 |
| | | EDTA | | |
| 10-3 | 5.6 ppm | 0.5 wt % Tris- | S. aureus | >6.8 |
| | | EDTA | | |
| 10-6 | 5.6 ppm | 0.5 wt % Tris- | P. aeruginosa | >7.4 |
| | | EDTA | | |

The results in Table 10 and in Table 4 show that whereas a tris-EDTA solution has no biocidal properties by itself, it decisively does not hinder and can synergistically assist in a broad-spectrum antimicrobial treatment.

### EXAMPLE 11

### Effect of UV (Sunlight)

Using the method of Example 9, varying amounts of toluidine blue or rose bengal dyes were added to water. The resulting compositions were exposed for 30 or 60 minutes to sunlight with and without a glass cover over the composition. Use of the glass cover screened out radiation below 300 nm. The compositions were evaluated for antimicrobial activity against S. aureus and P. aeruginosa. The results are set out below in Table 11.

TABLE 11
The Antimicrobial Effect of UV (Sunlight)

| | | | | Microbial | Microbial |
|---|---|---|---|---|---|
| | | | | Log | Log |
| | | | | Reduction | Reduction |
| Run | | Dye | | (30 | (60 |
| No. | Dye | Amount | Organism | Minute) | Minute) |
| 11-1 | Toluidine | 1.1 ppm | S. aureus | 4.3 | >6.3 |
| | Blue | | P. aeruginosa | >7.2 | >7.2 |
| 11-2 | Toluidine | 1.1 ppm | S. aureus | 3.9 | >6.3 |
| | Blue under | | P. aeruginosa | >7.2 | >7.2 |
| | glass | | | | |
| 11-3 | Rose | 3.3 ppm | S. aureus | >6.3 | >6.3 |
| | Bengal | | P. aeruginosa | >7.2 | >7.2 |
| 11-4 | Rose | 3.3 ppm | S. aureus | >6.3 | >6.3 |
| | Bengal | | P. aeruginosa | >7.2 | >7.2 |
| | under | | | | |
| | glass | | | | |

The results in Table 11 show that samples covered with glass exhibited antimicrobial efficacy that under these conditions was similar to that exhibited by samples that were not covered with glass. In general, exposure of the compositions to a broad spectrum of light containing both visible and UV radiation may provide enhanced antimicrobial efficacy.

Various modifications and alterations of this invention will be apparent to those skilled in the art without departing from the scope and spirit of the invention, and are intended to be within the scope of the following claims.

1. An antimicrobial composition comprising a singlet oxygen-generating agent and a lubricant.

2. A composition according to claim 1 in the form of an aqueous liquid.

3. A composition according to claim 1 in the form of a non-aqueous liquid.

4. A composition according to claim 1 in the form of a powder.

5. A composition according to claim 1 in the form of a shaped or molded solid.

6. A composition according to claim 1 wherein the agent comprises a photochemical singlet oxygen-generating agent.

7. A composition according to claim 1 wherein the singlet oxygen-generating agent comprises an aromatic carbonyl compound, condensed aromatic compound, acridine dye, coumarin dye, crystal violet, fluorene derivative, naphthalocyanine, porphyrin derivative, phthalocyanine, thiazine dye, thioketone, xanthene dye or mixture thereof.

8. A composition according to claim 1 wherein the singlet oxygen-generating agent comprises a xanthene dye, thiazine dye or mixture thereof.

9. A composition according to claim 1 wherein the singlet oxygen-generating agent comprises acetonapthone, acetophenone, benzophenone or a mixture thereof.

10. A composition according to claim 1 wherein the singlet oxygen-generating agent comprises acridine orange, eosin, fluorene, fluorenone, fluorescein, methylene blue, naphthalocyanine, phthalocyanine, rose bengal, thiocoumarin, toluidine blue, zinc tetraphenylporphyrin tetrasulfonate or a mixture thereof.

11. A composition according to claim 1 wherein the singlet oxygen-generating agent comprises rose bengal, methylene blue, toluidine blue, or a mixture thereof.

12. A composition according to claim 1 comprising about 0.1 to about 50 ppm of the singlet oxygen-generating agent based on the weight of the composition.

13. A composition according to claim 1 further comprising a chelating agent.

14. A composition according to claim 13 comprising about 0.1 to about 1000 ppm chelating agent based on the weight of the composition.

15. A composition according to claim 13 comprising about 1 to about 100 ppm chelating agent based on the weight of the composition.

16. A composition according to claim 1 further comprising a surfactant.

17. A composition according to claim 16 comprising about 1 ppm to about 10% surfactant based on the weight of the composition.

18. A composition according to claim 16 comprising about 10 ppm to about 1% surfactant based on the weight of the composition.

19. A composition according to claim 1 comprising ethylene diamine tetraacetate, tris(hydroxymethyl)aminomethane or a mixture thereof.

20. A method for antimicrobially lubricating the passage of a container along a conveyor comprising applying a lubricant to at least a portion of the container-contacting surface of the conveyor or to at least a portion of the conveyor-contacting surface of the container and generating singlet oxygen in situ.

21. A method according to claim 20 wherein the lubricant comprises an aqueous liquid.

22. A method according to claim 20 wherein the lubricant comprises a non-aqueous liquid.

23. A method according to claim 20 wherein the lubricant comprises a powder.

24. A method according to claim 20 wherein the lubricant comprises a shaped or molded solid.

25. A method according to claim 20 wherein singlet oxygen is generated photochemically.

26. A method according to claim 20 comprising applying a photo-activating dye to at least a portion of the container-contacting surface of the conveyor or to at least a portion of the conveyor-contacting surface of the container and exposing the dye to light to generate singlet oxygen.

27. A method according to claim 26 wherein the dye comprises an aromatic carbonyl compound, condensed aromatic compound, acridine dye, coumarin dye, crystal violet, fluorene derivative, naphthalocyanine, porphyrin derivative, phthalocyanine, thiazine dye, thioketone, xanthene dye or mixture thereof.

28. A method according to claim 26 wherein the dye comprises a xanthene dye, thiazine dye or mixture thereof.

29. A method according to claim 26 wherein the dye comprises acetonapthone, acetophenone, benzophenone or a mixture thereof.

30. A method according to claim 26 wherein the dye comprises acridine orange, eosin, fluorene, fluorenone, fluorescein, methylene blue, naphthalocyanine, phthalocyanine, rose bengal, thiocoumarin, toluidine blue, zinc tetraphenylporphyrin tetrasulfonate or a mixture thereof.

31. A method according to claim 26 wherein the dye comprises rose bengal, methylene blue, toluidine blue, or a mixture thereof.

32. A method according to claim 20 further comprising applying a chelating agent to at least a portion of the container-contacting surface of the conveyor or to at least a portion of the conveyor-contacting surface of the container.

33. A method according to claim 32 wherein the chelating agent comprises about 0.1 to about 1000 ppm of the weight of the lubricant.

34. A method according to claim 32 wherein the chelating agent comprises about 1 to about 100 ppm of the weight of the lubricant.

35. A method according to claim 20 further comprising applying a surfactant to at least a portion of the container-contacting surface of the conveyor or to at least a portion of the conveyor-contacting surface of the container.

36. A method according to claim 35 wherein the surfactant comprises about 1 ppm to about 10% of the weight of the lubricant.

37. A method according to claim 35 wherein the surfactant comprises about 10 ppm to about 1% of the weight of the lubricant.

38. A method according to claim 20 comprising applying ethylene diamine tetraacetate, tris(hydroxymethyl)aminomethane or a mixture thereof to at least a portion of the container-contacting surface of the conveyor or to at least a portion of the conveyor-contacting surface of the container.

39. A method according to claim 20 comprising irradiating the container or conveyor with sunlight, artificial room light, special lamps, or a combination thereof to generate singlet oxygen.

40. A method according to claim 20 wherein the singlet oxygen generation causes greater than a 1-log order reduction in the population of bacteria of a target species within 60 minutes at 23° C.

41. A lubricated conveyor or container, having a lubricant coating on a container-contacting surface of the conveyor or on a conveyor-contacting surface of the container, wherein the coating comprises a singlet oxygen-generating antimicrobial agent.

42. A conveyor or container according to claim 41, wherein the coating is in the form of an aqueous liquid.

43. A conveyor or container according to claim 41, wherein the coating is in the form of a non-aqueous liquid.

44. A conveyor or container according to claim 41, wherein the coating is in the form of a solid.

45. A conveyor or container according to claim 41, wherein the singlet oxygen-generating antimicrobial agent comprises a photochemical singlet oxygen-generating agent.

46. A conveyor or container according to claim 41, wherein the singlet oxygen-generating antimicrobial agent comprises an aromatic carbonyl compound, condensed aromatic compound, acridine dye, coumarin dye, crystal violet, fluorene derivative, naphthalocyanine, porphyrin derivative, phthalocyanine, thiazine dye, thioketone, xanthene dye or mixture thereof.

47. A conveyor or container according to claim 41, wherein the singlet oxygen-generating antimicrobial agent comprises a xanthene dye, thiazine dye or mixture thereof.

48. A conveyor or container according to claim 41, wherein the singlet oxygen-generating antimicrobial agent comprises acetonapthone, acetophenone, benzophenone or a mixture thereof.

49. A conveyor or container according to claim 41, wherein the singlet oxygen-generating antimicrobial agent comprises acridine orange, eosin, fluorene, fluorenone, fluorescein, methylene blue, naphthalocyanine, phthalocyanine, rose bengal, thiocoumarin, toluidine blue, zinc tetraphenylporphyrin tetrasulfonate or a mixture thereof.

50. A conveyor or container according to claim 41, wherein the singlet oxygen-generating antimicrobial agent comprises rose bengal, methylene blue, toluidine blue, or a mixture thereof.

51. A conveyor or container according to claim 41, wherein the singlet oxygen-generating antimicrobial agent comprises about 0.1 to about 50 ppm of the weight of the lubricant.

52. A conveyor or container according to claim 41, wherein the coating comprises a chelating agent.

53. A conveyor or container according to claim 51, wherein the chelating agent comprises about 0.1 to about 1000 ppm of the weight of the coating.

54. A conveyor or container according to claim 51, wherein the chelating agent comprises about 1 to about 100 ppm of the weight of the coating.

55. A conveyor or container according to claim 41, wherein the coating comprises a surfactant.

56. A conveyor or container according to claim 54, wherein the surfactant comprises about 1 ppm to about 10% of the weight of the coating.

57. A conveyor or container according to claim 54, wherein the surfactant comprises about 10 ppm to about 1% of the weight of the coating.

58. A conveyor or container according to claim 41, wherein the coating comprises ethylene diamine tetraacetate, tris(hydroxymethyl)aminomethane or a mixture thereof.
